(19) United States
(12) Patent Application Publication
(10) Pub. No.: US 2012/0141553 A1
(43) Pub. Date: Jun. 7, 2012

(54) BIOCOMPATIBLE POLYMER FIBRES FOR NEUROIMPLANTS

(76) Inventors: Abdellah Ajji, Mount-Royal (CA); Mahmud Bani, Ottawa (CA); Marianna Sikorska, Navan (CA)

(21) Appl. No.: 13/321,294

(22) PCT Filed: Jun. 30, 2010
(86) PCT No.: PCT/CA2010/001019

§ 371 Date: Nov. 18, 2011

(30) Foreign Application Priority Data
Jun. 30, 2009 (US) 61213665

(51) Int. Cl. A61K 31/765; A61P 25/00; D02G 3/00; A61K 35/30; A61K 35/50; A61K 9/70; A61K 35/12

(52) U.S. Cl. 424/400; 424/78.37; 428/364

(57) ABSTRACT

The present invention relates to a neuroimplant. The neuroimplant comprises biocompatible polymer fibres; the polymer fibres are grouped in a parallel arrangement, and the group of fibres is flexible. The present invention also relates to the use of the neuroimplant to facilitate the repair of damaged brain tissue.

### FIELD OF THE INVENTION

The present invention relates to biocompatible polymer fibres for neuroimplants. More specifically, the present inventions relates to biocompatible parallel polymer fibres for neuroimplants.

### BACKGROUND OF THE INVENTION

Brain injury and stroke are leading causes of death and disability worldwide (Green and Shuaib 2006; International Brain Injury Association, 2008). In Canada and the US, brain injury and stroke affect approximately 2 million people every year, of which more than 300,000 individuals die and at least another 300,000 end up with disabilities. The survivors join the current 10 million individuals who suffer from the chronic consequences of brain injury and stroke (Stroke Recovery Canada, 2004; Brain Injury Association of Nipissing: BIAN, 2005; International Brain Injury Association, 2007; Stroke Facts from Genetech, 2007). Disabilities include problems with sensory processing, motor function, communication, cognition, and mental health. In addition, a significant percentage of people who survive stroke are at the risk of another stroke. In many cases, strokes increase the risk for Alzheimer's disease, Parkinson's disease and other brain disorders that become more prevalent with age (Centre for Chronic Disease Prevention and Control Canada, 2008; National Institute of Neurological Disorders and Stroke, NINDS, 2006; Wen et al., 2008).

The treatments available for brain injury patients are very limited and include stabilization, monitoring, surgery and rehabilitation, depending on the case. In particular, surgical treatments are used to prevent secondary injury by helping to maintain blood flow and oxygen to the brain and minimize inflammation and pressure. While the bleeding inside the skull cavity is removed or drained, an intracranial pressure monitoring device may be placed surgically to supervise and control pressure. In cases of extensive injuries caused unintentionally or through surgical procedures to remove tumours, the damaged or diseased tissue is removed to make space for the living brain tissue. As a result, the neurons located in the damaged region lose their connections with the rest of the brain and need to functionally reconnect to prevent neurophysiological and cognitive problems. In many cases, the cavity left by the excised tumour is filled with absorbable hemostat (an oxidized regenerated cellulose product manufactured by Johnson & Johnson) to reduce inflammation. However, the commercially available hemostats do not facilitate neuroregeneration.

An extensive list of growth factors, neurotrophic factors, cytokines and drugs has also been explored as potential therapies. However, only a limited number of them may actually have the potential to effectively offset the brain injury or stroke-related problems. Common approaches to treatment of stroke include blood thinner medications, blood clot-dissolving drugs (such as recombinant tissue plasminogen activator, rt-PA), endarterectomy, and other surgeries. However, rt-PA must be administered within three hours of stroke, which excludes more than 95% of patients; furthermore, rt-PA does not provide reperfusion, and it increases the risk of symptomatic intracranial haemorrhage (Green and Shuaib 2006). Other neuroprotective drugs that reduce damage following brain injury or stroke have also been tested; however, none has been able to demonstrate efficacy in clinical trials (Marklund et al., 2006).

The efficient delivery of the right factor in a clinically-relevant time window may improve functional recovery after brain injury or stroke. Among commercially-available products, bone morphogenetic proteins (BMPs) are considered as one of the most promising candidates due to their role in modulating tissue repair and their long history of safe application in other diseases. To date, a few studies have suggested that stroke and other brain diseases may also benefit from BMP7. For instance, the intracisternal or intracerebroventricular administration of BMP7 improves motor function for at least two weeks after ischemia in rodents (Kawabata et al., 1998; Ren et al., 2000; Chou et al., 2006). However, multiple injections are required, possibly due to the short half life of BMP7 (10-30 minutes).

Cell implantation, in general, has been explored in the animal models of brain injury and stroke, and in a limited number of clinical trials (Borlongan et al. 1998; Kondziolka et al. 2000; Kelly et al. 2004; Lindvall et al. 2004; Muller et al. 2006; Wieloch and Nikolich 2006; Lindvall and Kokaia 2006). Clinical trials have shown the safety and feasibility of exogenous teratocarcinoma-derived neurons NT2N, mesenchymal stromal cells (MSC) and endothelial progenitors in stroke patients (Kondziolka et al., 2000; Kondziolka et al., 2005; Bang et al., 2005; Yip et al., 2008). Furthermore, they have shown functional synaptic communication between host brain and NT2N graft. These trials have been complemented by genetic modification of NT2N and MSC to deliver specific growth factors in the stroke animal models (Watson et al., 2003; Longhi et al., 2004; Horita et al., 2006; Zhao et al., 2006; Hara et al., 2008). There is also evidence that human fetal neural stem cells can enhance functional recovery by secreting glial cell line-derived neurotrophic factor (GDNF) in rats suffering form traumatic brain injury (Gao et al., 2006).

Several studies have shown that the adhesion, survival and proliferation of neural cells require an appropriate microenvironment (Park et al., 2002; Teng et al., 2002; Bani-Yaghoub et al. 2005). To achieve regeneration and functional reconnectivity, implants must fill the gaps in the brain tissue formed during phagocytosis of dying cells and scar tissue formation. While the injection of cells into the damaged region may partially reduce the gap size, many cells must be injected to fill the gap after injury; of these cells, many die or fail to functionally connect to the host tissue.

Cells seeded on synthetic biocompatible polymers seem to have the advantage of a more permissive environment for connectivity. So far, a number of polymers have been successfully used to generate reciprocal interactions between graft and host in the post-stroke cortex, the Parkinson's disease striatum, injured visual cortex and injured spinal cord (Sautter et al. 1998; Park et al., 2002; Teng et al., 2002; Ahn et al. 2005; Tatard et al. 2007). Among these polymers, polylactic acid (PLA), polyglycolic acid (PGA) and polylactic-co-glycolic acid (PLGA) have been approved by the Food and Drug Administration (FDA) and demonstrate optimal mechanical strength, biocompatibility and biodegradability (Bueno et al. 2007). PLA, PGA, and PLGA have successfully been used in reconstructive surgery to repair damaged peripheral nerves (such as facial, digital and plantar nerves) in patients, and have shown promise as synthetic nerve guides (Schlosshauer et al., 2006). In addition to nerve guides, commercially-available polymer mesh (PGA mesh, Japan) have been used to repair incidental dural tears in patients (Shimada et al. 2006). However, neither the design nor the dimensions of nerve guides is suitable for regeneration of damaged brain tissue.

These problems continue to encourage new research to further understand the mechanisms by which neurons are formed, and to develop novel strategies that promote brain repair.

### SUMMARY OF THE INVENTION

The present invention relates to biocompatible polymer fibres for neuroimplants. More specifically, the present inventions relates to flexible biocompatible parallel polymer fibres for neuroimplants.

In one aspect, the present invention provides a neuroimplant comprising biocompatible polymer fibres, wherein the polymer fibres are grouped in a parallel arrangement, and wherein the group of fibres are flexible. The fibres of the neuroimplant just described may be formed from thermoplastic material. For example, the fibres may be poly(glycolic acid) fibres, polylactic acid fibres, or a combination thereof. The polymer fibres within the meuroimplant may also be in substantial contact with one another.

The neuroimplants may further comprise cells that facilitate the regeneration of brain tissue. Such cells may be embryonic stem cells, neural stem cells, neural progenitors, NT2 cells, amniotic fluid cells, amniotic fluid stem cells, blood cord cells, or a combination thereof. The cells may be engineered to deliver neurotrophic, neuroprotective, or neuroregenerative factors to the brain. The factors may include glial cell line-derived neurotrophic factor (GDNF) and/or bone morphogenetic protein 7 (BMP7), or a combination thereof.

The present invention further encompasses a method of facilitating the repair of damaged brain tissue, comprising placing a neuroimplant as described herein in the damaged area, and allowing the regeneration of neurons to occur. The neuroimplant may additionally comprise cells that facilitate the regeneration of brain tissue, which may or may not be engineered to deliver neurotrophic factors, neuroprotective factors, or neuroregenerative factors, or a combination thereof to the brain (as described above). The method as described may further comprise a step of inducing the expression of the neurotrophic factors, neuroprotective factors, and/or neuroregenerative factors.

The neuroimplant as described above may provide a template for cell attachment, survival, proliferation and differentiation, neurite growth, tissue reconstitution/regeneration and functional connectivity and recovery. The topological features of the implant may facilitate the reconstruction of damaged brain after injury, stroke or tumour excision, by serving as a template to reconnect the injured brain tracts.

Neuroimplants in accordance with the present invention support cell adhesion and survival. Seeding of mouse embryonic stem (ES) cells, neural stem (NS) cells, neural progenitors (NP) and neuroblasts, and human NT2 cells on neuroimplants of the present invention shows that these cells can differentiate into neurons on the neuroimplants. Neurites from these cell types followed the pattern of PGA fibres by extending along the fibres. Furthermore, the production of specific factors by these cells as well as human amniotic fluid (AF) cells carried by the neuroimplants of the present invention was confirmed by ELISA and other methods. Also, the neuroimplants presently described were shown to have a beneficial effect in the regeneration of mouse motor cortex following injury.

Additional aspects and advantages of the present invention will be apparent in view of the following description. The detailed description and examples, while indicating preferred embodiments of the invention, are given by way of illustration only, as various changes and modifications within the scope of the invention will become apparent to those skilled in the art in light of the teachings of this invention.

### BRIEF DESCRIPTION OF THE DRAWINGS

These and other features of the invention will now be described by way of example, with reference to the appended drawings, wherein:

FIG. 1A is a perspective view of a portion of a neuroimplant in accordance with the present invention. The neuroimplant is flat and is comprised of parallel polymer fibres. FIG. 1B is a perspective view of a neuroimplant of the present invention where the polymer fibres are formed to a C-shape. FIG. 1C shows another embodiment of the neuroimplant of the present invention, having multiple layers. Cells may be grown on and between fibres of the present neuroimplant. FIG. 1D shows a Hoffman modulation contrast image of a neuroimplant prepared in accordance with the present invention.

FIG. 2A shows a schematic of the BMP7 lentiviral vector. FIG. 2B shows confirmation of BMP7 transgene expression by fluorescence microscopy 18 hours after transfecting the packaging HEK 293SF-PacLv cells. Scale bar: 50 μm. FIG. 3 shows the BMP7-Lentivirus titration and protein production for non-infected 293GPG cells (FIG. 3A); 1:100 BMP7-Lv infected 293GPG cells (FIG. 3B); 1:10 BMP7-Lv infected 293GPG cells (FIG. 3C); and 1:1 BMP7-Lv infected 293GPG cells (FIG. 3D). FIG. 3E is a bar graph showing that at least 75% of the cells were infected with BMP7 lentivirus at 1:1 dilution. FIG. 3F is a western blot of the infected HEK 293GPG cultures showing production of BMP7 protein. BMP7 protein was present in the cultures as early as 48 hours following infection. Samples included: mouse cerebrospinal fluid (lane 1), cells infected with GFP-Lv (lane 2), medium from BMP7 lentivirus infected cultures (lane 3), medium (10× concentrated) from GFP-Lv infected cultures (lane 4), medium (10x concentrated) from BMP7 lentivirus infected cultures (lane 5).

FIG. 4 shows that BMP7 is consistently produced and released into the medium from approximately 1×10⁶ BMP7-Lv infected 293 GPG cells. FIGS. 4A and B show ELISA results for cells 3 and 28 days after infection, respectively. FIG. 4C is a bar graph showing the amount of BMP7 secreted over a 24-hour period, in nanograms; approximately 350 ng of BMP7 is secreted into the media every 24 hours. FIG. 4D shows western blot analysis of the biological activity of BMP7 protein produced by lentiviral system (Lv-BMP7) compared to that of commercially available recombinant human BMP7 (rBMP7). Lane 2: primary embryonic day 13 (E13) cortical progenitor cells treated with GFP-Control media; Lanes 3:1 ng/mL of rBMP7, Lanes 4-5: Lv-BMP7. FIG. 4E is a bar graph showing that, similar to recombinant human BMP7 (rhBMP7), there was a significant increase in the number of MAP2 positive neurons in the embryonic day 13 (E13) cortical progenitor cultures treated with the lentivirally-made BMP7 (Lv-BMP7) for 5 days (*, ** p<0.001).

FIG. 5A shows seeding of mouse N2a cells on neuroimplants. Both N2a (FIG. 5B) and mouse embryonic stem (ES) cells (FIGS. 5C-D) can differentiate into neurons on neuroimplants. Both N2a and ES cells have been stained with the cell survival dye, 5CFDA.

FIGS. 6A and B show GFP-tagged human amniotic fluid cells grown on neuroimplants.

FIG. 6C shows human amniotic fluid cells tagged with GDNF-GFP, while FIG. 6D shows human amniotic fluid cells tagged with BMP7-GFP.

FIG. 7 shows high resolution digital photographs of the healthy (FIG. 7A) and injured (FIG. 7B, circled) brains. Corresponding immunohistochemical images show intact neurons (arrowheads) in the healthy motor cortex (FIG. 7C) and neurons affected by injury (FIG. 7D), showing MAP2 immunoreactivity. Cb: cerebellum, Ncx: neocortex, OB: olfactory bulb. *: lost tissue, Scale bar: A and B 1.6 mm, C and D 70 μm.

FIG. 8 shows tissue reconstitution in the motor cortex after receiving a neuroimplant.

FIG. 8A shows an adult mouse left motor cortex (arrow) two months after injury, having received no cell or polymer implantation); the right motor cortex has been used as control.

FIG. 8B shows the left motor cortex (arrow) one month after injury and implantation with the neuroimplant (PGA polymer+cells) of the present invention; the right motor cortex (asterisk) is 15 minutes post-injury was used as an internal control.

### DETAILED DESCRIPTION OF THE INVENTION

The present invention relates to biocompatible polymer fibres for neuroimplants. More specifically, the present invention relates to flexible biocompatible parallel polymer fibres for neuroimplants.

In one aspect, the present invention provides a neuroimplant comprising biocompatible polymer fibres, wherein the polymer fibres are grouped in a parallel arrangement, and wherein the group of fibres are flexible.

The neuroimplant of the present invention, also referred to herein as “neural implant” or “implant”, is intended for implantation into brain tissue. The present neuroimplant has topological features that facilitate the reconstruction of damaged brain after injury, stroke or tumour excision, by serving as a template to reconnect the injured brain tracts.

The neuroimplant of the present invention is comprised of biocompatible polymer fibres. By the term “biocompatible”, it is meant that the fibres are compatible for placement in a living system or tissue; “biocompatible” also indicates that the polymer fibres can integrate with the tissue without eliciting an immune response in the organism.

By the term “polymer fibres”, it is meant a synthetic material that is a continuous filament. The polymer fibres are synthesized from chemical moieties using physical processes well-known in the art. The polymer fibres used in the present invention may be a single polymer, a co-polymer, or blend of polymers. The neuroimplant may comprise a number of fibres, wherein individual fibres may be made of the same or different materials.

The polymer fibres may be biodegradable or non-degradable. A biodegradable polymer fibre may be degraded within a time interval that is compatible for neuroregeneration of the brain; this time interval may depend on the size and severity of the damage. For example, and without wishing to be limiting in any manner, the polymer fibres may be substantially degraded in 5 to 15 weeks; for example, the polymer fibres may be substantially degraded in 5, 6, 7, 8, 9, 10, 11, 12, 13, 14, or 15 weeks, or any time there between, or within a range of times defined by any two values just recited.

The polymer fibres may be made of any suitable material, including but not limited to: polyester; polyethylene; polymethacrylic; polyacrylic; polysulfone; polyurethane; nylon (polyamide); aliphatic polyesters; poly(amino acids); copoly(ether-esters); polyalkylene oxalates; polyamides; poly(iminocarbonates); polyorthoesters; polyoxaesters; polyamidoesters; poly(anhydrides); polyphosphazenes; polyphosphoester; and biopolymers. In a non-limiting example, the polymer fibres may be polylactic acid (PLA) fibres, for example poly(L-lactic acid) or poly(DL-lactic acid); poly(glycolic acid) (PGA) fibres; polylactic-co-glycolic acid (PLGA) fibres; polycaprolactone polyanhydride fibres; chitosan fibres; sulfonated chitosan fibres; polyglycolide fibers; poly-4-hydroxybutyrate fibres; or polyphosphoester fibres. In a specific, non-limiting example, polymer fibres may be formed from thermoplastic material; the polymer fibres may be PGA and/or PLA fibres.

The size of the polymer fibres in the neuroimplant of the present invention may be any size suitable for regeneration of brain tissue. The polymer fibres may have a diameter of about 5 to about 120 microns; for example, the diameter of the fibres may be 5, 10, 15, 20, 25, 30, 35, 40, 45, 50, 55, 60, 65, 70, 75, 80, 85, 90, 95, 100, 105, 110, 115 or 120 microns, or any size therebetween, or any range of sizes defined by any two values just recited. The neuroimplant of the present invention may comprise polymer fibres of the same diameter, or of varying diameters. As would be recognized by a person of skill in the art, the length of the polymer fibres would vary based on the physical requirements of the neuroimplant.

The neuroimplant of the present invention may comprise a suitable number of polymer fibres. Without wishing to be limiting in any manner, the neuroimplant may comprise 5-500 polymer fibres; for example, the neuroimplant may comprise 5, 10, 15, 20, 25, 30, 35, 40, 45, 50, 75, 100, 125, 150, 175, 200, 225, 250, 275, 300, 325, 350, 375, 400, 425, 450, 475, or 500 polymer fibres, or any amount therebetween. The amount of fibres within the implant may vary based on the type of polymer used, as well as the size of the fibres; the amount of fibres in the neuroimplant may be determined by a skilled person based on these variables.

The size of the implant, the diameter of the fibres, the number of fibres, the type of polymer(s) and the rate of degradation of the neuroimplant of the present invention may be adjusted in accordance with the physical requirements of the particular application. As would be understood by a person of skill in the art, polymer type, molecular weight, and blend may be adjusted in order to address the needs of the application at hand.

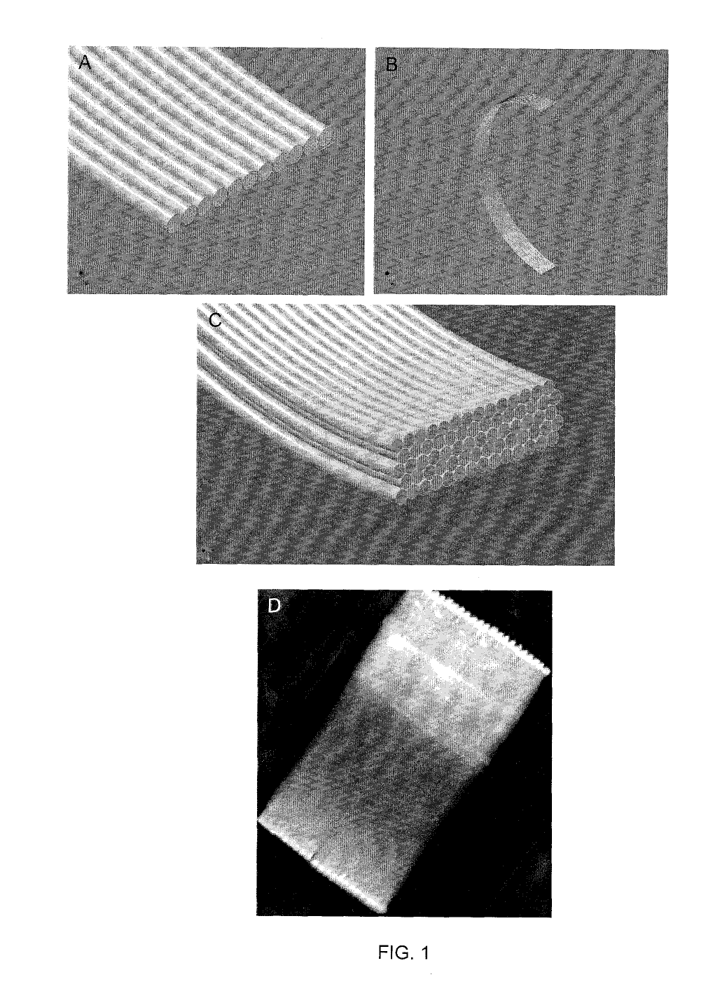

Importantly, the polymer fibres of the neuroimplant are in a parallel arrangement. By the term “parallel arrangement”, it is meant that the long axes (also referred to herein as “length”) of the fibres are placed parallel to each other (see FIG. 1A). This feature differs from the currently used polymer mesh (Shimada et al., 2006), which has randomly-oriented fibres that lack the architecture or topology required to reconnect damaged brain tracts. Without wishing to be limiting, the parallel arrangement and proper orientation of the polymer fibres in the neuroimplant of the present invention presents regular features that may allow neurons to attach, grow and expand linearly; this may allow the neurons to communicate and link with each other and may provide improved conditions for neurite growth.

Furthermore, the fibres in parallel arrangement must be in substantial contact with one another. By “substantial contact”, it is meant that the fibres contact each other along at least part of their length on at least one side. While some areas of non-contact are permissible, these must not interfere with the overall design or integrity of the neuroimplant. Areas of non-contact may be located at regular intervals, or at varying intervals along the length of the neuroimplant. The polymer fibres may be bonded or consolidated together to maintain contact between each other; the bonding may be permanent. The fibres may be bonded together using any suitable method known in the art. For example, and without wishing to be limiting in any manner, gradually heating thermoplastic fibres above their glass transition temperature, but before complete flow, followed by cooling would allow them to be bonded together. Bonding of the fibres should not alter the arrangement, configuration or shape of the fibres or the neuroimplant.

The polymer fibres may be grouped (also referred to herein as “bundled”) together in various configurations, provided they remain in a parallel arrangement. For example, and without wishing to be limiting in any manner, the polymer fibres may be grouped in a monolayer of bonded fibres (see for example, FIG. 1A), in multiple layers bonded fibres (see for example, FIG. 1C), in a cylinder (hollow or filled), or any other suitable configuration. These configurations, together with the parallel arrangement of the fibres, create channels between the fibres that may encourage regeneration of neurons.

The group of fibres in the neuroimplant of the present invention may be flexible. By the term “flexible”, it is meant that the group(s) of fibres may be formed into a desired geometry or shape. The desired shape may vary based on the area of the brain tissue receiving the implant and/or the type of implant required. Generally, the implant may be required to be flat, to be curved, or to include curved sections along its length. For example, and without wishing to be limiting in any manner, the group of fibres may be formed into a flat implant, or one that is C-shaped (see FIG. 1B), U-shaped, S-shaped, J-shaped, semi-cylindrical, or any other suitable shape. The group of fibres may be shaped using any suitable method known in the art. For example, and without wishing to be limiting in any manner, the group of fibres may be formed into the desired shape along its length during the bonding process described above; in this non-limiting example, thermoplastic fibres are heated while in contact with a mandrel to form the fibres into the desired shape (mandrel shape). For example, flat or curved shapes may be obtained using a flat plate or cylinder, respectively, on which the fibres are rolled, then consolidated or bonded under heat. Once formed into the desired shape, the group of fibres retains the shape after removal from the mandrel. Non-limiting examples of shapes of neuroimplants of the present invention are shown in FIG. 1.

The neuroimplants of the present invention may further comprise cells that facilitate the regeneration of brain tissue. As would be recognized by one of skill in the art, the type of cells to be used in conjunction with the neuroimplant will vary based on the organism receiving the implant. For example, and without wishing to be limiting in any manner, the cells may be mouse embryonic stem cells, mouse neural stem cells, mouse neural progenitors, mouse N2a cells, human embryonic stem cells, human neural stem cells, human neural progenitors, NT2 cells (including NT2 differentiated cells such as NT2 neurons and astrocytes), human amniotic fluid cells, human amniotic fluid stem cells, human blood cord cells, or any other suitable type of cell. In a specific, non-limiting example, the cells may be embryonic stem cells, neural stem cells, neural progenitors, NT2 cells, amniotic fluid cells, amniotic fluid stem cells, blood cord cells, or a combination thereof.

The cells may be engineered to deliver neurotrophic factors, neuroprotective factors, or neuroregenerative factors, or a combination thereof to the brain. For example, and without wishing to be limiting in any manner, the cells may be genetically engineered to produce one or more than one factor known to be involved in tissue repair following the implantation; for example, the factors may be glial cell line-derived neurotrophic factor (GDNF) and/or bone morphogenetic protein 7 (BMP7). The production and the amount of factor(s) secreted by the engineered cells may be regulated. This regulation may be achieved by any suitable method known in the art. For example and without wishing to be limiting in any manner, an inducible lentiviral delivery system may be used to regulate factor expression in these cells under a tetracycline (Tet)-responsive bi-directional promoter; this allows for tight regulation of factor expression, thus enabling controlled delivery.

The present invention also encompasses a method of facilitating the repair of damaged brain tissue, comprising placing a neuroimplant as described above in the damaged area, and allowing the regeneration of neurons to occur. The neuroimplant may additionally comprise cells that facilitate the regeneration of brain tissue, which may or may not be engineered to deliver neurotrophic factors, neuroprotective factors, or neuroregenerative factors, or a combination thereof to the brain (as described above). The method as described may further comprise a step of inducing the expression of the neurotrophic factors, neuroprotective factors, and/or neuroregenerative factors.

The neuroimplant as described above may provide a template for cell attachment, survival, proliferation and differentiation, neurite growth, tissue reconstitution/regeneration and functional connectivity and recovery. The topological features of the implant may facilitate the reconstruction of damaged brain after injury, stroke or tumour excision, by serving as a template to reconnect the injured brain tracts.

Neuroimplants in accordance with the present invention support cell adhesion and survival. Seeding of various neural cell types (see above) on neuroimplants of the present invention shows that cells can differentiate into neurons on the neuroimplants. Neurites from both cell types followed the pattern of PGA fibres by extending along the fibres. The production of specific factors by cells carried by the neuroimplants of the present invention was confirmed by ELISA and other methods. Also, the neuroimplants presently described were shown to have a beneficial effect in the regeneration of mouse motor cortex following injury.

The present invention will be further illustrated in the following examples. However, it is to be understood that these examples are for illustrative purposes only and should not be used to limit the scope of the present invention in any manner.

### EXAMPLE 1

### Preparation of the Polymer Fibre Neuroimplant

A neuroimplant in accordance with the present invention was prepared as described below.

Purasorb PG (PURAC), a polyglycolic acid (PGA), was used for the preparation of the neuro-implant, due to its degradation time characteristics (within a few weeks). First, fibres of various diameters (5 to 120 microns) were produced from PGA using a capillary rheometer in combination with a rotating wheel winder. The barrel temperature was set at 280° C. and the fibre was formed at room temperature to allow for very fast cooling and to avoid crystallization. Differential scanning calorimetric analysis showed that the fibres were completely amorphous (data not shown). The fibres were stored at −18° C. after production.

The neuroimplant was produced by rolling a long PGA fibre around either a metallic plate or cylinder (“mandrel”). The implants produced had dimensions of about 3 mm in length. Once the fibres were closely rolled around the mandrel, they were subjected to high temperature (about 210° C.) either in an air convection oven or using a hot air stream on the surface of the fibres such that only the fibre surface was melted. The exposure time to high temperature (with continuous rotation of the mandrel) was about 5 minutes and depended on the desired degree of bonding. A Hoffman modulation contrast image of a prepared neuroimplant is shown in FIG. 1D.

### EXAMPLE 2

### Construction of Lentiviral Vectors

An inducible lentiviral delivery system was prepared for BMP7 expression in cells under a tetracycline (Tet)-responsive bi-directional promoter.

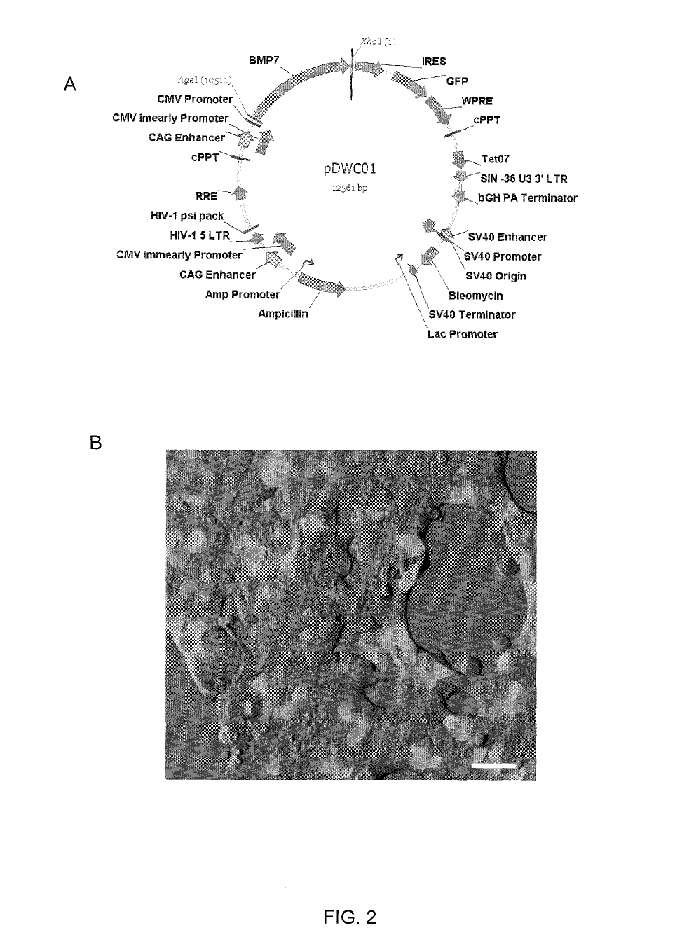

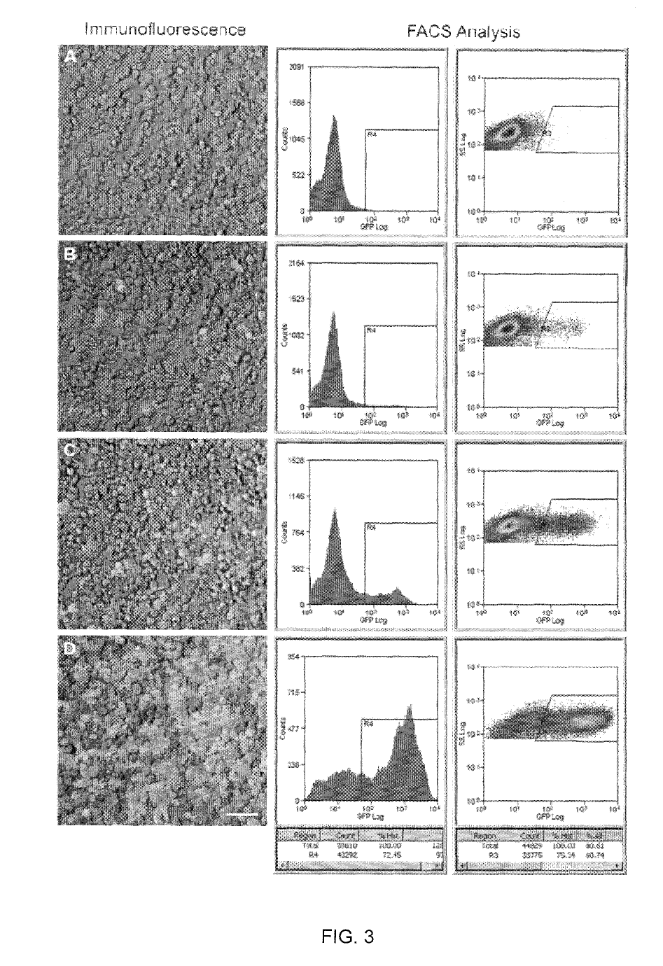

A safe and efficient lentiviral vector, pTet07CSII-CMV-GFPq (kindly provided by Dr. Bernard Massie, NRC-BRI, (Broussau et al., 2008)) was utilized for cloning. The plasmid pDWC01 was constructed through standard cloning procedures and isolated with Qiagen MaxiPrep kit. Briefly, the sequence encoding BMP7 was cut from pCMV-SPORT6-BMP7 (Open Biosystems) with the restriction endonucleases AgeI and XhoI. The vector pTet07CSII-CMV-GFPq was linearized with AgeI and XhoI to form compatible ends for ligation. To construct the lentiviral BMP7 vector (pDWC01), the cut BMP7 DNA fragment was ligated (T4 DNA ligase, NEB) into pTet07CSII-CMV-GFPq, upstream of an Internal Ribosomal Entry Site and Green Fluorescent Protein (IRES-GFP). The resulting plasmid encoded for a third generation transfer lentivector with the transgenes BMP7 and GFP under the control of a CMV promoter (FIG. 2A). Similar techniques were used to make GDNF-GFP lentiviral vector (Sandhu et al., 2009). Both BMP7 and GDNF inserts were sequenced to ensure their accuracy.

### EXAMPLE 3

### Isolation of Neural Stem and Neural Progenitor Cells

Neural stem and neural progenitor cells were isolated from mice, in preparation for transfection and implantation.

Timed-pregnant mice were sacrificed by CO₂ inhalation at embryonic day 13 (E13), according to a protocol approved by the NRC-IBS Animal Care Committee (ACC), as previously described (Bani-Yaghoub et al., 2006). The uteruses were aseptically removed and transferred sequentially to two Petri dishes containing calcium- and magnesium-free Hank's balanced salt solution (HBSS, Invitrogen Corporation, Burlington, ON) to rinse away blood. Embryos were dissected out of the amniotic sacs and examined for morphological hallmarks to ensure the accuracy of the gestational timing. The heads and the telencephalons were sequentially isolated under a dissection microscope and transferred into the new plates containing HBSS. The dorsal and ventral telencephalic regions were dissected out and freed of meninges and dissected further to isolate the ventricular zone (VZ).

Tissues were mechanically dissociated in Dulbecco's Modified Eagle Medium, high glucose, L-glutamine (DMEM; Invitrogen) and filtered through a 40 μm nylon cell strainer (Falcon, VWR, Mississauga, ON). The dissociated cells were quickly assessed for viability by the trypan blue exclusion assay. Neural stem cells were examined for the self-renewal and multipotential properties, using neurosphere assays (Bani-Yaghoub et al., 2006). In brief, cells were deposited into the uncoated 96-well plates (Nunc) in DMEM (Invitrogen)+N2 supplement (Invitrogen)+fibroblast growth factor 2 (FGF2, 20 ng/ml, Invitrogen) at a density of 1 cell/well (plating efficiency: ˜40%). Single cells were repeatedly monitored under a light microscope for the neurosphere formation, using the same culture condition. Neurospheres were dissociated with trypsin and transferred onto the PLL-coated neuroimplants in DMEM+5% fetal bovine serum (FBS)+N2 supplement and examined 1-10 days later for the expression of neuronal markers. Neural progenitors were obtained from the E13.5 VZ and seeded directly onto the PLL-coated neuroimplants and treated with DMEM+5% fetal bovine serum (FBS)+N2 supplement.

### EXAMPLE 4

### Transduction of Cells with the GDNF- or BMP7-IRES-GFP Lentivirus

The lentiviral delivery system of Example 2 was introduced to cells, yielding cells that express GDNF and/or BMP7.

The 293SF-PacLV packaging cells were seeded in 10 cm dishes and transfected with the plasmid pDWC01 (3^{rd} generation lentivirus encoding BMP7 or GDNF and control green fluorescent protein (GFP)), using Lipofectamine 2000 (Invitrogen) (Broussau et al., 2008). Six hours after transfection, medium was replaced with fresh medium supplemented with 1 μg/ml doxycycline and 10 μg/ml cumate (4-Isopropylbenzoic acid). The medium containing lentivirus was harvested at 72 h after transfection, filtered with 0.45 μm filters and concentrated with Amicon Ultra-15 spin columns (100,000 mol. wt. cut off, Millipore). Then, the virus was applied to neural progenitors, including amniotic fluid cells, after which the transduced cells were selected (Bani-Yaghoub et al., 2006; Sandhu et al., 2009).

The sample results of FIG. 2B confirm BMP7 transgene expression by fluorescence microscopy 18 hours after transfecting the packaging HEK 293SF-PacLv cells.

### EXAMPLE 5

### FACS-Based Titration and Lentiviral Infection

The fluorescent-activated cell sorting (FACS)-analysis was used to determine the transducing units (TU)/mL of BMP7-Lv or GDNF produced by transfected 293SF cells (Example 4) 48 hrs post-transfection.

Briefly, HEK 293GPG cells were seeded in six-well plates at a density of 1.0E⁶ cells/well and incubated at 37° C. in 5% CO2 for 24 hrs or until cells were approximately 85-90% confluent (˜2.0E⁶ cells/well). To remove potential cell debris prior to infection, the medium was replaced with 1.7 mL/well of fresh DMEM with 1% FBS. Serial dilutions were prepared with DMEM in the ratios 1:1, 1:10 and 1:100 from 30× concentrated lentiviral-containing medium. Each 293GPG-containing well was transduced with 300 μL of the desired lentiviral serial preparation. Polybrene was added to a final concentration of 8 μg/mL for each the control and the infection wells and the plates were subsequently incubated at 37° C. in 5% CO₂. Following a 48 hr incubation period, the infection efficiency was verified with fluorescent microscopy via the examination of GFP expression. The cells were prepared for FACS analysis, first by removing the control and infection medium from each well and washing with 1× phosphate-buffered saline (PBS). Next, 200 μL of 0.25% Trypsin was added to each well and following a short 1 min incubation period at RT, the cells were resuspended in 1 mL/well of PBS containing 10% FBS, briefly vortexed to dissociate the cells and stored on ice. An aliquot of the sample was counted using a hemocytometer to determine the approximate cell density per well. The samples were immediately analyzed on a MoFlo flow cytometer (DakoCytomation, Copenhagen, Denmark) using Summit software. For each sample at least 40,000 events were collected. The titer of the virus was determined using the following formula: transducing units/ml=[(% Infected Cells)×(Total Cell Number in Well)×(Dilution Factor)]/(Volume of Inoculum Added to Cells).

FIGS. 3A-D show the BMP7-lentivirus titration via FACS analysis of non-infected 293GPG cells, 1:100 BMP7-Lv infected 293GPG cells, 1:10 BMP7-Lv infected 293GPG cells, and 1:1 BMP7-Lv infected 293GPG cells, respectively. These results show that at least 75% of the cells were infected with BMP7 lentivirus at 1:1 dilution (FIG. 3E). A western blot of the infected HEK 293GPG cultures (FIG. 3F) indicates that BMP7 was present in the cultures as early as 48 hours following infection.

### EXAMPLE 6

### BMP7 and GDNF ELISA

The level of BMP7 and GDNF proteins expressed by the cells of Example 4 was quantified using a human BMP7 or GDNF ELISA development kit, according to the manufacturer's protocol (R&D Systems, Minneapolis, Minn., USA).

BMP7: Briefly, 96-well flat-bottomed Maxisorp plates (Nunc International) were coated with the capture antibody (mouse anti-human BMP7 capture antibody) diluted 1:180 with 1× PBS, pH 7.2 and incubated overnight at room temperature (RT). Following overnight incubation, the wells were blocked for 1 hr at room temperature with 200 μL of Reagent Diluent (PBS+1% BSA, pH 7.2) per well. Standards for BMP7, ranging from a low of 125 pg/mL to a high of 8000 pg/mL were prepared using recombinant human BMP7 (R&D Systems) diluted in Reagent Diluent and the samples were prepared in serial dilutions (1:1, 1:10, 1:100) with PBS. Approximately 100 μL/ well of each standard and sample dilution were applied to the plate in duplicate and incubated at RT for 2 hrs. The wells were washed 5× with 200 μL/well of Wash Buffer (PBS, 0.05% (v/v) Tween 20, pH 7.2) followed by the addition of 100 μL/well of BMP7 detection antibody (biotinylated mouse anti-human BMP7 antibody) diluted 1:180 in Reagent Diluent+2% heat-inactivated goat serum. Following a 2 hr incubation period at RT and another wash step, 100 μL of streptavidin-conjugated horseradish peroxidase (streptavidin-HRP, R&D Systems) diluted in Reagent Diluent (1:200) was applied to each well and incubated at RT for 20 min. The wells were again washed (5×) with Wash Buffer and color development was achieved by adding 100 μL of a 1:1 mixture of tetramethylbenzidine (TMB; Sigma-Aldrich, Oakville, Ontario): H₂O₂ per well. The plates were incubated for 20 min at room temperature in the dark and the reaction was stopped by the addition of 50 μL 2 N HCl per well. The absorbance was measured using a SpectraMax 340 microplate reader (Molecular Devices, Sunnyvale, Ca, USA) at 450 nm and the amount of BMP7 was calculated from the standard curves in the detection limit range.

GDNF: The amount of GDNF released in HAF cultures transduced with Lenti-GDNF or Lenti-GFP was measured using a GDNF Eₘₐₓ® Immunoassay system according to the manufacturer's instructions (Promega, Madison, Wis.). In brief, Maxisorp 96-well, flat-bottomed ELISA plates (Nalgene Nunc International) were coated with anti-GDNF monoclonal antibody diluted in carbonate coating buffer, pH 8.2 and incubated overnight at 4° C. Wells were blocked for 1 hour at room temperature with 1× blocking buffer (200 μL/well). GDNF standards ranging from 0-1000 pg/100 μL were prepared using recombinant human GDNF and sample dilutions (100 μL, dilutions ranging from 5-fold to 20-fold) were applied to the wells. All samples were incubated with shaking for 6 hours at room temperature and then washed with TBS-T (20 mM Tris-HCl, pH 7.6, 150 mM NaCl, 0.05% (v/v) Tween 20). The captured GDNF was bound by a specific polyclonal antibody on incubating overnight at 4° C. After washing, the amount of bound polyclonal antibody specific to GDNF was then detected by a species specific (chicken) antibody conjugated to horse radish peroxidase incubated overnight at 4° C. Following washes with TBS-T, horseradish peroxidase-conjugated anti-chicken IgY antibody was added to the plates and incubated with shaking at room temperature for 2 hours. The plates were again washed with TBS-T, and 100 μL of the enzyme substrate (Tetramethylbenzidine One solution) was added. The plates were incubated for 15 min at room temperature in the dark and the reaction was stopped by the addition of 100 μL 1N HCl per well. The absorbance was measured at 450 nm and the amount of GDNF was calculated from the standard curve in the linear range.

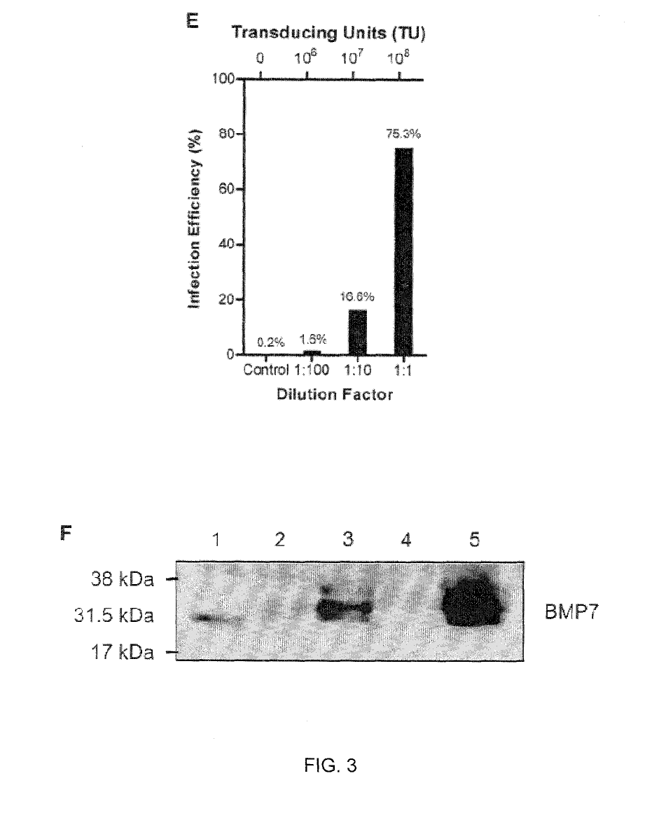

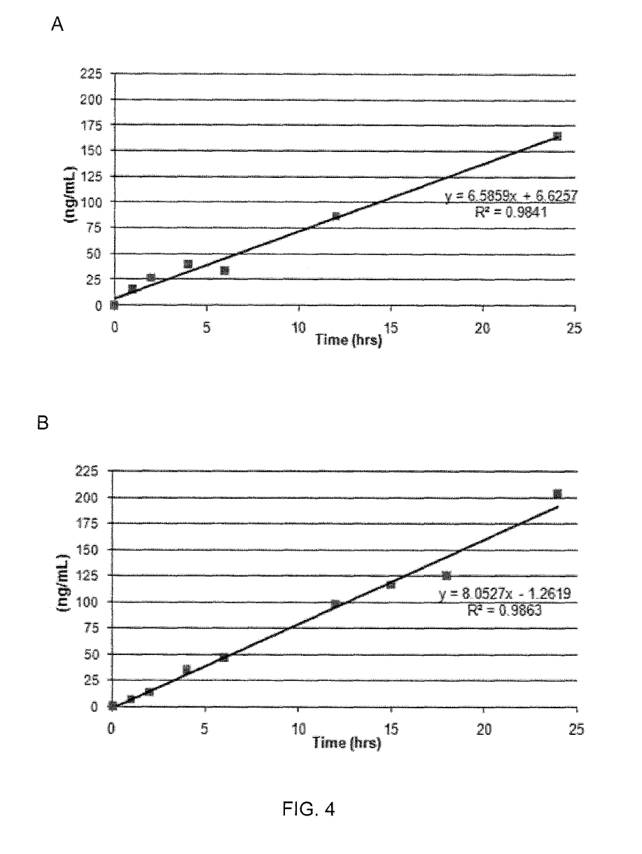

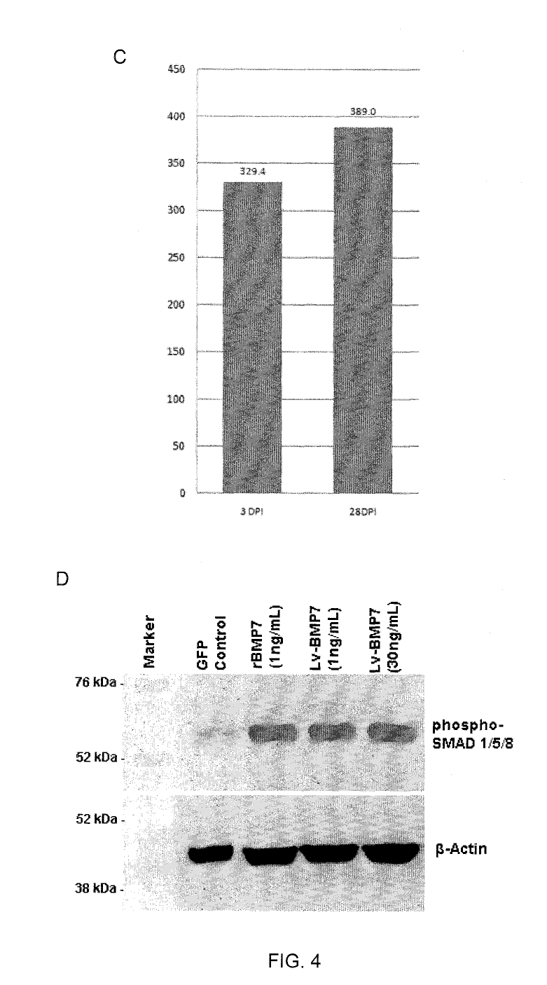

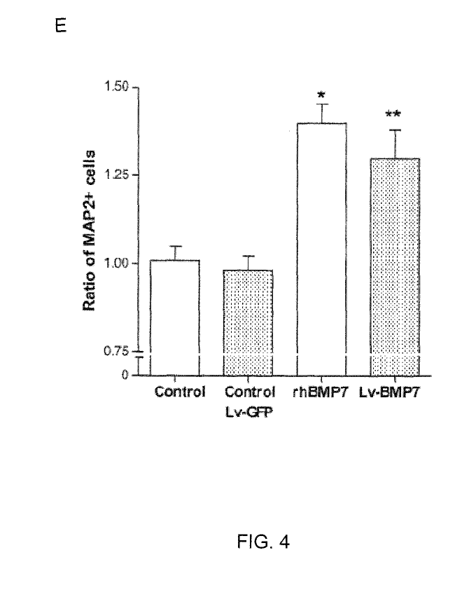

ELISA results are shown in FIGS. 4A-4C. The level of BMP7 secretion was markedly high in BMP7-Lv infected 293GPG cultures. After 3 days, the level of BMP7 secreted by 1×10⁶ cells was up to 330 ng over a 24-hr period. To determine the long-term BMP7 producing capacity of the infected cultures, the level of BMP7 was determined 4 weeks following infection. The level of BMP7 was consistent 4 weeks later with a maximum yield of 390 ng of BMP7 secreted over a 24-hr period. The biological activity of the BMP7 protein produced by lentiviral system (Lv-BMP7) was verified by comparing with that of the commercially available recombinant human BMP7 (FIG. 4D). In brief, primary embryonic day 13 (E13) cortical progenitor cells were treated with GFP-Control media (lane 2), 1 ng/mL of rBMP7 or Lv-BMP7 (lanes 3 and 4) and 30 ng/mL Lv-BMP7 (lane 5) for 1.5 hrs to examine SMAD 1/5/8 activation and translocation to the nucleus.

Using similar ELISA methods, approximately, 10 ng of GDNF was secreted from 1×10⁶ human amniotic fluid (AF) cells within 24 hours. Both BMP7 and GDNF were consistently produced and released into the media. Additionally, results (FIG. 4E) show that there was a significant increase in the number of MAP2 positive neurons in the embryonic day 13 (E13) cortical progenitor cultures treated with the lentivirally-made BMP7 (Lv-BMP7) for 5 days.

### EXAMPLE 7

### Neuroimplant Seeding and Evaluation

To construct neuroimplants, cells (mouse or human ES, NS, NP, NT2 or AF) were seeded on the scaffolds.

Initially, seeding was done in the presence of Dulbecco's Modified Eagle Medium (DMEM)+10% fetal bovine serum (FBS), and then in DMEM+0.5% FBS+N2 supplement (i.e., prior to implantation). While the size of the neuroimplant and cell density are easily adjustable, cells were seeded at a density of 2.5×10³−1×10⁵ cells on neuroscaffolds that approximate the size of 2.5 week old male C57BL/6 mouse primary motor cortex (I: 3 mm×w: 2 mm×1 mm).

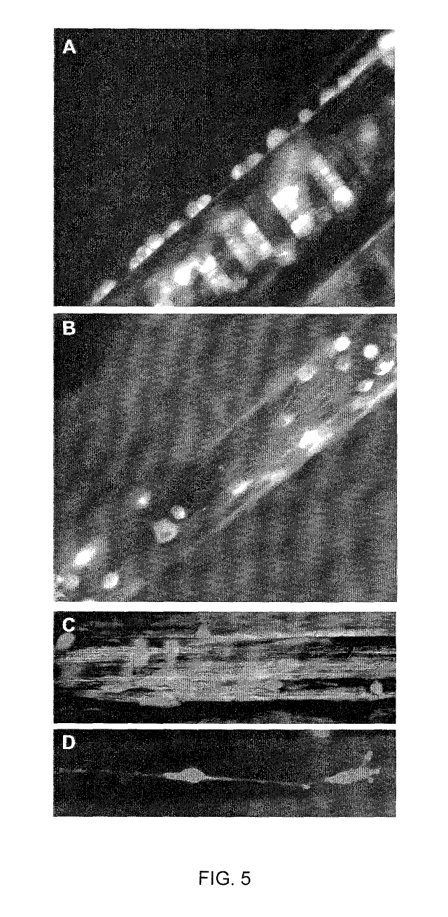

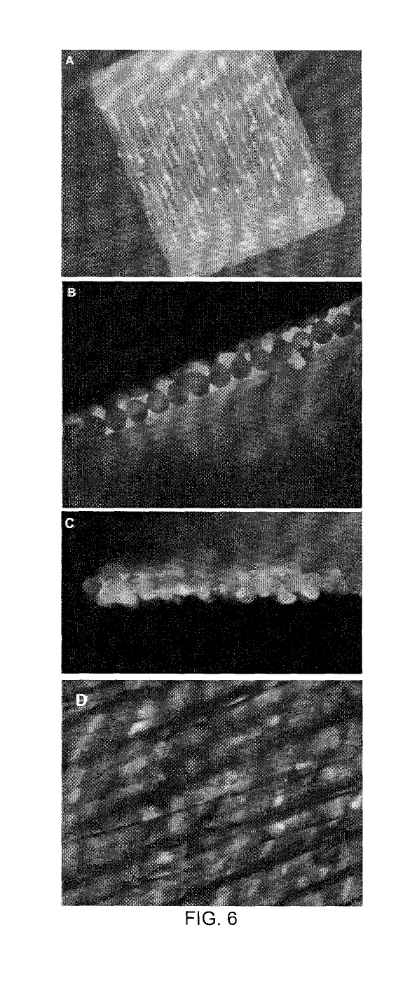

FIG. 5 shows results of the seeding of N2a and mouse embryonic stem cells on the neuroimplant of the present invention. Both N2a (FIG. 5B) and mouse embryonic stem (ES) cells (FIGS. 5C-D) can differentiate into neurons on neuroimplants, and neurites from both cell types follow the pattern of PGA scaffold by extending along the scaffold fibres. Thus, it is presently shown that the neuroimplant design allows the formation of organized neurite growth. FIGS. 6 show that cells can grow on neuroimplants and secrete neurotrophic/neuroprotective/neuroregenerative factors; specifically, the GFP (FIGS. 6A-B), GFP-GDNF (FIG. 6C), and BMP7-GFP human amniotic fluid cells (Example 4) were grown on neuroimplants. The production of GDNF factors by cells was confirmed by ELISA and other methods (see Example 6).

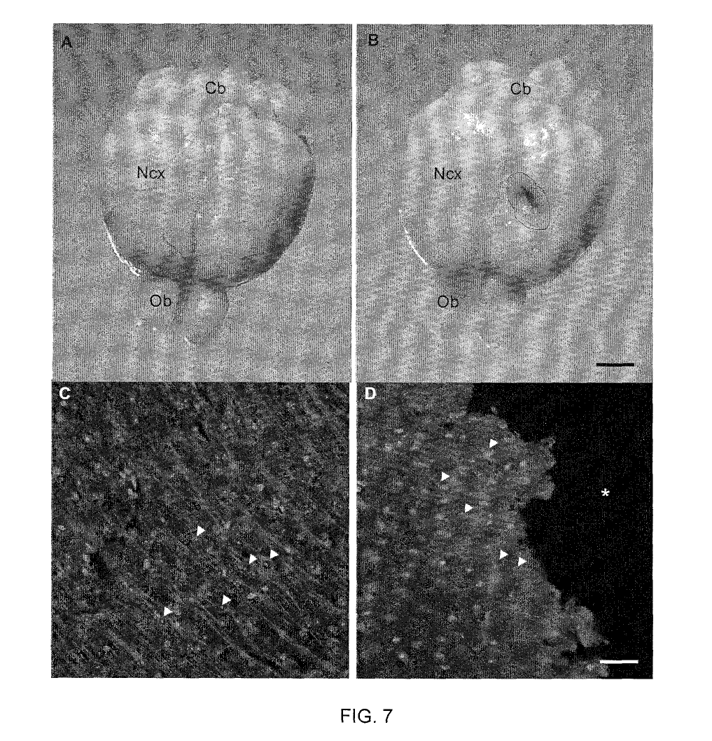

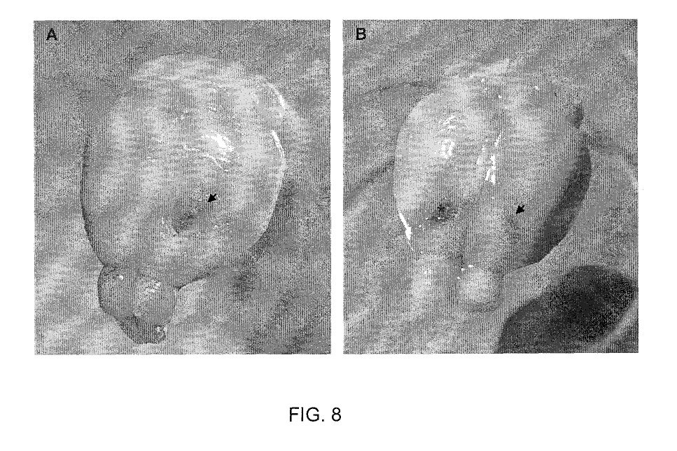

The in vivo performance of the neuroimplant of the present invention was also evaluated. Injury was mechanically introduced to the left motor cortex of adult mouse brains (FIG. 7B, circled, and FIG. 8A). In brief, 56-77 day old C57BI/6 or CD1 mice (Charles River Labs, St Constant, QC) were anesthetizedusing isoflurane gas (Aerrane, Baxter, Montreal, QC). The animals were placed in a stereotaxic frame and the skull was exposed. The injury site was marked on the bone, using specific coordinates (from Lat +0.7 mm, AP −0.25 mm to −1.0 mm to Lat +2.4 mm AP +1.25 mm to +3.0 mm) and the bone was removed with a dental drill. The motor cortex was injured, using a sterile graduated needle/knife to the depth of 1 mm (DV 1 mm). FIG. 7 shows images of healthy (FIG. 7A) and injured (FIG. 7B) adult mouse brains. In addition to the control non-injured mice (FIG. 7A), the right motor cortex was used as internal control (non-injured hemisphere in FIGS. 7B and 8A). Corresponding immunohistochemical images show intact neurons (arrowheads) in the healthy motor cortex (FIG. 7C). In contrast, neurons are significantly affected by injury, as evidenced by morphological features and MAP2 immunoreactivity (FIG. 7D). A representative image of the left motor cortex that had not received cell or polymer implantation (FIG. 8A, arrow) has been shown two months after injury. In another case, the left motor cortex received the PGA polymer neuroimplant seeded with cells (see above) and was evaluated one month after injury (FIG. 8B, arrow). To better compare the significance of the repair in the left motor cortex after implantation (FIG. 8B, arrow), an acute injury was introduced to the right motor cortex of the same mouse 15 minutes before the brain was taken out (FIG. 8B, denoted by asterisk).

Together, FIG. 8 shows tissue reconstitution in the motor cortex after receiving a neuroimplant of the present invention. In the absence of any implantation, the injured adult mouse left motor cortex shows little improvement 2 months post-injury. In contrast, implantation of the neuroimplant (PGA polymer+cells) of the present invention in the left motor cortex shows significant regeneration of the brain tissue one month post-injury.

The embodiments and examples described herein are illustrative and are not meant to limit the scope of the invention as claimed. Variations of the foregoing embodiments, including alternatives, modifications and equivalents, are intended by the inventors to be encompassed by the claims. Furthermore, the discussed combination of features might not be necessary for the inventive solution.

### REFERENCES

All patents, patent applications and publications referred to herein are hereby incorporated by reference.

Ahn, Y. H., Bensadoun, J. C., Aebischer, P., Zurn, A. D., Seiger, A., Bjorklund, A., Lindvall, O., Wahlberg, L., Brundin, P., Kaminski, Schierle, G. S. 2005. Increased fiber outgrowth from xeno-transplanted human embryonic dopaminergic neurons with co-implants of polymer-encapsulated genetically modified cells releasing glial cell line-derived neurotrophic factor. Brain Res Bull 66:135-142.

Bang S M, Kim Y K, Park Y H, Sohn S K, Lee J J, Cho E K, Ryoo B Y, Chung I J, Yoon S S, Kim H J, Lee J H, Yoon H J, Park S. 2005. High-dose therapy and autologous stem cell transplantation in Korean patients with aggressive T/NK-cell lymphoma. Leuk Lymphoma. 11:1599-1604.

Bani-Yaghoub M, Tremblay R, Voicu R, Mealing G, Monette R, Py C, Faid K, Sikorska M. 2005. Neurogenesis and neuronal communication on micropatterned neurochips. Biotechnol Bioeng 92:336-345.

Bani-Yaghoub, M., Tremblay, R. G., Lei, J. X., Zhang, D., Zurakowski, B., Sandhu, J. K., Smith, B., Ribecco-Lutkiewicz, M., Kennedy, J., Walker, P. R. and Sikorska, M. (2006) Role of Sox2 in the development of the neocortex. Dev Biol 295:52-66.

Bian C, Song X, Liu Z, Zhang H. 2005. Design proposal of imaging activities of cultured neural network on a silicon substrate with neural-electronic-optical integrated microsystem. Conf Proc IEEE Eng Med Biol Soc. 7:7600-3.

Borlongan C V, Saporta S, Sanberg P R. 1998. Intrastriatal transplantation of rat adrenal chromaffin cells seeded on microcarrier beads promote long-term functional recovery in hemiparkinsonian rats. Exp Neurol. 2:203-14.

Brain Injury Association of Nipissing: BIAN (2005) http://dawn.thot.net/brain/Broussau S, Jabbour N, Lachapelle G, Durocher Y, Tom R, Transfiguracion J, Gilbert R, Massie B. 2008. Inducible packaging cells for large-scale production of lentiviral vectors in serum-free suspension culture. Mol Ther. 3:500-7.

Bueno, E M., Laevsky, G., Barabino, G. A. 2007. Enhancing cell seeding of scaffolds in tissue engineering through manipulation of hydrodynamic parameters. J Biotechnol 129:516-531.

Centre for Chronic Disease Prevention and Control Canada (2008) http://www.phac-aspc.gc.ca/ccdpc-cpcmc/

Chang, C. F., Lin, S. Z., Chiang, Y. H., Morales, M., Chou, J., Lein, P., Chen, H. L., Hoffer, B. J., Wang, Y. 2003. Intravenous administration of bone morphogenetic protein-7 after ischemia improves motor function in stroke rats. Stroke 34:558-564.

Chou, J., Harvey, B. K., Chang, C. F., Shen, H., Morales, M., Wang, Y. 2006. Neuroregenerative effects of BMP7 after stroke in rats. J Neurol Sci 240:21-29.

De Coppi P, Callegari A, Chiavegato A, Gasparotto L, Piccoli M, Taiani J, Pozzobon M, Boldrin L, Okabe M, Cozzi E, Atala A, Gamba P, Sartore S. 2007. Amniotic fluid and bone marrow derived mesenchymal stem cells can be converted to smooth muscle cells in the cryo-injured rat bladder and prevent compensatory hypertrophy of surviving smooth muscle cells. J 1:369-76.

Gao J, Prough DS, McAdoo D J, Grady J J, Parsley M O, Ma L, Tarensenko Y I, Wu P. 2006. Transplantation of primed human fetal neural stem cells improves cognitive function in rats after traumatic brain injury. Exp Neurol. 2:281-92.

Green A R, Shuaib A. 2006. Therapeutic strategies for the treatment of stroke. Drug Discov Today. 15-16:681-93.

Hara K, Yasuhara T, Maki M, Matsukawa N, Masuda T, Yu S J, Ali M, Yu G, Xu L, Kim S U, Hess D C, Borlongan C V. 2008. Neural progenitor NT2N cell lines from teratocarcinoma for transplantation therapy in stroke. Prog Neurobiol. 3:318-34.

Horita Y, Honmou O, Harada K, Houkin K, Hamada H, Kocsis J D. 2006. Intravenous administration of glial cell line-derived neurotrophic factor gene-modified human mesenchymal stem cells protects against injury in a cerebral ischemia model in the adult rat. J Neurosci Res. 2006 Nov 15;84(7):1495-504

International Brain Injury Association, 2007. http://www.internationalbrain.org/

International Brain Injury Association, 2008. http://vvvvw.internationalbrain.org/

Kawabata, M., Imamura, T., Miyazono, K. 1998. Signal transduction by bone morphogenetic proteins. Cytokine Growth Factor Rev 9:49-61.

Kelly, S., Bliss, T. M., Shah, A. K., Sun, G. H., Ma, M., Foo, W. C., Masel, J., Yenari, M. A., Weissman, I. L., Uchida, N., Palmer, T., Steinberg, GK. 2004. Transplanted human fetal neural stem cells survive, migrate, and differentiate in ischemic rat cerebral cortex. Proc Natl Acad Sci USA 101:11839-11844.

Kondziolka, D., Wechsler, L., Goldstein, S., Meltzer, C., Thulborn, K. R., Gebel, J., Jannetta, P., DeCesare, S., Elder, E. M., McGrogan, M., Reitman, M. A., Bynum, L. 2000. Transplantation of cultured human neuronal cells for patients with stroke. Neurology 55:565-569.

Kondziolka D, Steinberg G K, Wechsler L, Meltzer C C, Elder E, Gebel J, Decesare S, Jovin T, Zafonte R, Lebowitz J, Flickinger J C, Tong D, Marks M P, Jamieson C, Luu D, Bell-Stephens T, Teraoka J. 2005. Neurotransplantation for patients with subcortical motor stroke: a phase 2 randomized trial. J Neurosurg. 1:38-45.

Longhi L, Watson D J, Saatman K E, Thompson H J, Zhang C, Fujimoto S, Royo N, Castelbuono D, Raghupathi R, Trojanowski J Q, Lee V M, Wolfe J H, Stocchetti N, McIntosh T K. 2004. Ex vivo gene therapy using targeted engraftment of NGF-expressing human NT2N neurons attenuates cognitive deficits following traumatic brain injury in mice. J Neurotrauma. 12:1723-36.

Lindvall, O., Kokaia, Z., Martinez-Serrano, A. 2004. Stem cell therapy for human neurodegenerative disorders-how to make it work. Nat Med 10 Suppl:S42-50.:S42-S50.

Lindvall, O., Kokaia, Z. 2006. Stem cells for the treatment of neurological disorders. Nature 441:1094-1096.

Marklund N, Bakshi A, Castelbuono D J, Conte V, McIntosh T K. 2006. Evaluation of pharmacological treatment strategies in traumatic brain injury. Curr Pharm Des. 13:1645-80.

Muller, F. J., Snyder, E. Y., Loring, J. F. 2006. Gene therapy: can neural stem cells deliver? Nat Rev Neurosci 7:75-84.

National Institute of Neurological Disorders and Stroke, NINDS, 2006 www.ninds.nih.gov

Park, K. I., Teng, Y. D., Snyder, E. Y. 2002. The injured brain interacts reciprocally with neural stem cells supported by scaffolds to reconstitute lost tissue. Nat Biotechnol 20:1111-1117.

Ren, J., Kaplan, P. L., Charette, M. F., Speller, H., Finklestein, S. P. 2000. Time window of intracisternal osteogenic protein-1 in enhancing functional recovery after stroke. Neuropharmacology. 39:860-5.

Sandhu J K, Gardaneh M, lwasiow R, Lanthier P, Gangaraju S, Ribecco-Lutkiewicz M, Tremblay R, Kiuchi K, Sikorska M. 2008. Astrocyte-secreted GDNF and glutathione antioxidant system protect neurons against 6OHDA cytotoxicity. Neurobiol Dis. 3:405-14.

Sautter, J., Sabel, M., Sommer, C., Strecker, S., Weidner, N., Oertel, W. H., Kiessling, M. 1998. BDNF and TrkB expression in intrastriatal ventral mesencephalic grafts in a rat model of Parkinson's disease. J Neural Transm 105:253-263.

Schlosshauer, B., Dreesmann, L., Schaller, H. E., Sinis, N. 2006. Synthetic nerve guide implants in humans: a comprehensive survey. Neurosurgery 59:740-747.

Shimada Y, Hongo M, Miyakoshi N, Sugawara T, Kasukawa Y, Ando S, Ishikawa Y, Itoi E. 2006. Dural substitute with polyglycolic acid mesh and fibrin glue for dural repair: technical note and preliminary results. J Orthop Sci. 5:454-8

Simic, P., Vukicevic, S. 2007. Bone morphogenetic proteins: from developmental signals to tissue regeneration. Conference on bone morphogenetic proteins. EMBO Rep 8:327-331.

Stroke Facts from Genetech, 2002.

www. gene .comlgenelproductsleducation/vascularlstroke-factsheet.html

Stroke Recovery Canada (2004) http://www.strokerecoverycanada.com/

Tatard, V. M., Sindji, L., Branton, J. G., Ubert-Pouessel, A., Colleau, J., Benoit, J. P., Montero-Menei, C. N. 2007. Pharmacologically active microcarriers releasing glial cell line—derived neurotrophic factor: Survival and differentiation of embryonic dopaminergic neurons after grafting in hemiparkinsonian rats. Biomaterials 28:1978-1988.

Teng, Y. D., Lavik, E. B., Qu, X., Park, K. I., Ourednik, J., Zurakowski, D., Langer, R., Snyder, E. Y. 2002. Functional recovery following traumatic spinal cord injury mediated by a unique polymer scaffold seeded with neural stem cells. Proc Natl Acad Sci USA 99:3024-3029.

Watson D J, Longhi L, Lee E B, Fulp C T, Fujimoto S, Royo N C, Passini M A, Trojanowski J Q, Lee V M, McIntosh T K, Wolfe J H. 2003. Genetically modified NT2N human neuronal cells mediate long-term gene expression as CNS grafts in vivo and improve functional cognitive outcome following experimental traumatic brain injury.

J Neuropathol Exp Neurol. 4:368-80.

Wen, H., Dou, Z., Finni, T., Havu, M., Kang, Z., Cheng, S., Sipila, S., Sinha, S., Usenius, J. P., Cheng, S. 2008. Thigh muscle function in stroke patients revealed by velocity-encoded cine phase-contrast magnetic resonance imaging. Muscle Nerve, March 11.

Wieloch, T., Nikolich, K. 2006. Mechanisms of neural plasticity following brain injury. Curr Opin Neurobiol 16:258-264.

Yip S, Shah K. 2008. Stem-cell based therapies for brain tumors. Curr Opin Mol Ther. 4:334-42.

Zhao B, Cooper L J, Brahma A, MacNeil S, Rimmer S, Fullwood N J. 2006. Development of a three-dimensional organ culture model for corneal wound healing and corneal transplantation.Invest Ophthalmol Vis Sci. 7:2840-6.

1. A neuroimplant comprising biocompatible polymer fibres, wherein the polymer fibres are grouped in a parallel arrangement, and wherein the group of fibres is flexible.

2. The neuroimpiant of claim 1, wherein the polymer fibres are in substantial contact with one another.

3. The neuroimplant of claim 1, wherein the fibres are formed from thermoplastic material.

4. The neuroimplant of claim 3, wherein the fibres are poly(glycolic acid) fibres, polylactic acid fibres, or a combination thereof.

5. The neuroimplant of claim 1, further comprising cells that facilitate regeneration of brain tissue.

6. The neuroimplant of claim 5, wherein the cells are embryonic stem cells, neural stem cells, neural progenitors, NT2 cells, amniotic fluid cells, amniotic fluid stem cells, blood cord cells, or a combination thereof.

7. The neuroimplant of claim 5, wherein the cells are engineered to deliver neurotrophic factors, neuroprotective factors, neuroregenerative factors, or a combination thereof to the brain.

8. The neuroimplant of claim 7, wherein the cells are engineered to deliver glial cell line-derived neurotrophic factor (GDNF), bone morphogenetic protein 7 (BMP7), or a combination thereof.

9. A method of facilitating the repair of damaged brain tissue, comprising placing the neuroimplant of claim 1 in the damaged area, and allowing the regeneration of neurons to occur.

10. The method of claim 9, wherein the neuroimplant further comprises cells that facilitate the regeneration of brain tissue

11. The method of claim 10, wherein cells are engineered to deliver neurotrophic factors, neuroprotective factors, or neuroregenerative factors, or a combination thereof to the brain.

12. The method of claim 11, further comprising a step of inducing the expression of the neurotrophic factors, neuroprotective factors, and/or neuroregenerative factors.
